(19) United States
(12) Patent Application Publication
(10) Pub. No.: US 2013/0046343 A1
(43) Pub. Date: Feb. 21, 2013

(54) Spinal Mobilization Device

(71) Applicants: Julie Adamski, Gilbertsville, PA (US); Dennis Adamski, Gilbertsville, PA (US)

(72) Inventors: Julie Adamski, Gilbertsville, PA (US); Dennis Adamski, Gilbertsville, PA (US)

(21) Appl. No.: 13/572,911

(22) Filed: Aug. 13, 2012

Related U.S. Application Data
(60) Provisional application No. 61/523,864, filed on Aug. 16, 2011.

(51) Int. Cl. A61F 5/00

(52) U.S. Cl. 606/240

(57) ABSTRACT

A device for mobilizing a joint includes a flat, planar substrate having a first face and an opposing second face. A first longitudinal side extends between the first face and the second face. A second longitudinal side extends between the first face and the second face, distal from the first longitudinal side. A mobilization portion extends above the first face and includes a first convex rib extending parallel to the first longitudinal side, a second convex rib extending along the second longitudinal side and parallel to the first convex rib, and a valley extending between the first convex rib and the second convex rib along a longitudinal centerline of the flat, planar substrate. A cavity extends above the first face. The cavity is defined by an opening between the first face and the mobilization portion and is sized to receive a bladder.

### CROSS-REFERENCE TO RELATED APPLICATION

The present application claims priority from U.S. Provisional Patent Application Ser. No. 61/523,864, filed on Aug. 16, 2011, which is incorporated herein by reference in its entirety.

### FIELD OF THE INVENTION

The present invention relates to a device and a method for mobilizing a spine during physical therapy.

### BACKGROUND OF THE INVENTION

Physical therapists and chiropractors manipulate different areas of a patient's body in order to relieve tension and stress, particularly along the patient's spinal column. Typically, the patient is treated by lying in either the supine or the prone position, with the caregiver applying pressure to the patient's body in order to manipulate the patient's spinal column. For a larger patient, however, the caregiver may not be able to perform a traditional joint mobilization because the caregiver cannot get a correct hand placement under the patient during treatment. Additionally, the amount of pressure used to manipulate a patient's spinal column may vary between different caregivers or even by the same caregiver at different times.

It would be beneficial to provide a device and a method for mobilizing a patient's spine or other joint while being able to determine the amount of pressure that is being applied to the joint and being able to replicate the pressure value in subsequent mobilization procedures.

### BRIEF SUMMARY OF THE INVENTION

Briefly, the present invention provides a device for mobilizing a joint comprising a flat, planar substrate having a first face and an opposing second face. A first longitudinal side extends between the first face and the second face. A second longitudinal side extends between the first face and the second face, distal from the first longitudinal side. A mobilization portion extends above the first face and includes a first convex rib extending parallel to the first longitudinal side, a second convex rib extending along the second longitudinal side and parallel to the first convex rib, and a valley extending between the first convex rib and the second convex rib along a longitudinal centerline of the flat, planar substrate. A cavity extends above the first face. The cavity is defined by an opening between the first face and the mobilization portion and is sized to receive a bladder.

Further, the present invention also provides a method for mobilizing a spine comprising the steps of placing a patient in one of a seated position and a sidelying position on a generally flat surface; applying the device described above such that the valley is aligned in a desired location along the patient's spine; reclining the patient to a supine position such that the device is located between the generally flat surface and the patient; and applying pressure to the patient, forcing the patient against the device.

Additionally, the present invention provides a device for mobilizing a joint comprising a flat, planar substrate having a first face and an opposing second face. A first longitudinal side extends between the first face and the second face and a second longitudinal side extends between the first face and the second face, distal from the first longitudinal side. A mobilization portion extends above the first face and comprises a first convex rib extending parallel to the first longitudinal side, a second convex rib extending along the second longitudinal side and parallel to the first convex rib, a valley extending between the first convex rib and the second convex rib along a longitudinal centerline of the flat, planar substrate, and a deflection measurement device configured to measure deflection of the mobilization portion toward the planar surface.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. The drawings are not necessarily drawn to scale. In the drawings:

FIG. 1 is a perspective view of a mobilization device according to a first exemplary embodiment of the present invention;

FIG. 2 is a top plan view of the mobilization device shown in FIG. 1;

FIG. 3 is a side view, in section, taken along lines 3-3 of FIG. 2;

FIG. 4 is a side view, in section, taken along lines 4-4 of FIG. 2;

FIG. 5 is a side view of the device of FIG. 1 being used with a patient;

FIG. 6 is a side view, in section, of a mobilization device according to a second exemplary embodiment of the present invention; and

FIG. 7 is a flowchart illustrating an exemplary method of using the mobilization devices of FIGS. 1-6.

### DETAILED DESCRIPTION OF THE INVENTION

In the drawings, like numerals indicate like elements throughout. Certain terminology is used herein for convenience only and is not to be taken as a limitation on the present invention. The term “longitudinal” refers to a longer portion of a planar substrate and the term “lateral” refers to a shorter portion of the planar substrate, generally orthogonal to the longitudinal portion. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

One aspect of the present invention includes a device that is used to mobilize a patient's spine during therapy or chiropractic adjustment. The inventive device is used as a fulcrum to manipulate a patient's thoracic and lumbar spine. The patient is laid in a supine position on a relatively hard surface, such as, for example, a massage or chiropractic table or a floor. The inventive device is located between the patient and the surface, at a desired location along the patient's spine. When the inventive device and the patient are both in a desired position, a caregiver, such as, for example, a physical therapist or a chiropractor, applies pressure to the patient's torso area, forcing the patient against the device. The caregiver may hold the inventive device in one of his/her hands and apply the pressure with the other forearm while leaning into the patient's chest. Alternatively, the caregiver may cross his/her arms across his/her chest and lean onto the patient, using more of his/her body weight to apply the desired amount of pressure to the patient. The caregiver increases the amount of pressure use on the patient until an audible cavitation or “pop” at the spine is heard.

The shape of the inventive device and its location along the patient's spine, along with the pressure applied by the caregiver, allow for mobilization of the patient's spine. It is interesting to note, however, for larger patients, the mere act of lying on the inventive device may be sufficient to mobilize the patient's spine, with the patient's own body weight applying the force necessary to mobilize his/her spine. While an exemplary use of the inventive device is to mobilize the patient's spine, those skilled in the art will recognize that the inventive device can also be used to mobilize other joints as well.

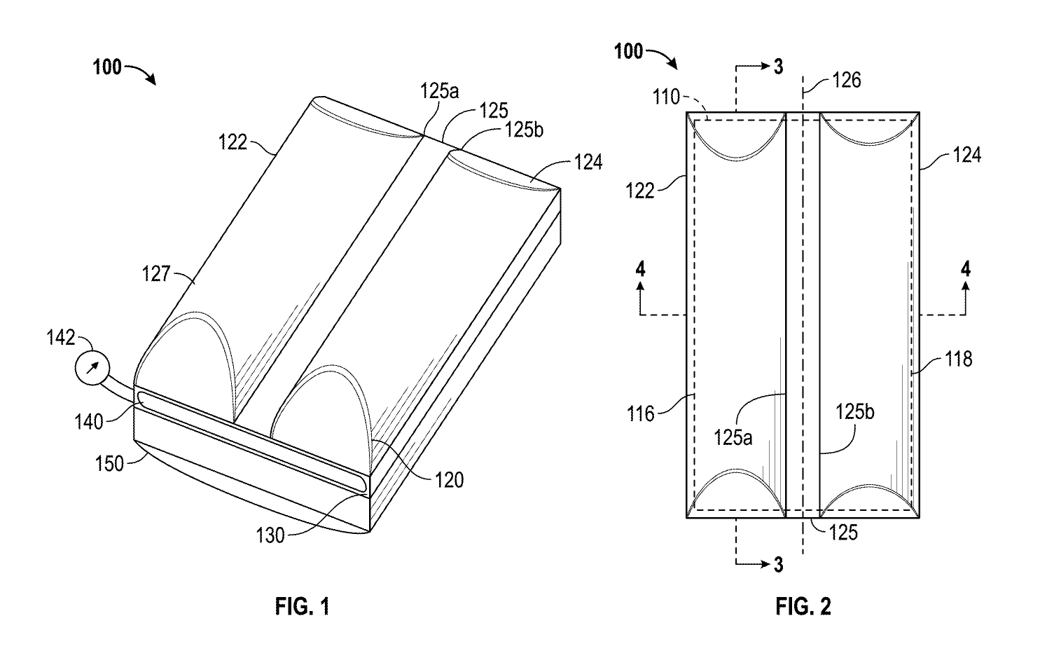

Referring in general to the figures and in particular, to FIGS. 1-4, a mobilization device 100 according to an exemplary embodiment of the present invention is shown. Mobilization device 100 includes a flat, planar substrate 110. In an exemplary embodiment, substrate 110 is a stiff and rigid material, such as, for example wood or plastic, and has an exemplary longitudinal dimension of about 4 inches (about 10.2 cm), an exemplary lateral dimension of about 3 inches (about 7.6 cm) and a thickness of about ⅛ inch (about 0.3 cm). The inventor believes that a longitudinal dimension of substrate 110 significantly greater than the exemplary longitudinal dimension of about 4 inches may actually result in an immobilization of the patient's spine, thereby possibly defeating the purpose of using the inventive device.

Substrate 110 includes a top, or first, face 112 and an opposing bottom, or second, face 114. A first longitudinal side 116, which is about ⅛ thick, extends between first face 112 and second face 114. A second longitudinal side 118, which is also about ⅛ inch thick, extends between first face 112 and the second face 114, distal from first longitudinal side 116.

A mobilization portion 120 extends upward above first face 112 and includes a first convex rib 122 that extends longitudinally across substrate 110 along and parallel to first longitudinal side 116. A second convex rib 124 extends longitudinally across substrate 110 along second longitudinal side 118 and parallel to first convex rib 122. A valley 125 extends between first convex rib 122 and second convex rib 124 along a longitudinal centerline 126 of substrate 110.

Each of first and second convex rib 122, 124 can be constructed from a fabric outer covering 127 and filled with a granular material 128, such as, for example, sand or other fine material, in order to provide a relatively firm, yet slightly pliable outer surface. Granular material 128 is located between fabric outer covering 127 and first face 112 of substrate 110. Alternatively, first and second convex rib 122, 124 can be constructed from a relatively hard, yet flexible, durometer material, such as, for example, a hard rubber, neoprene, low resistance polyurethane, or any other similar type of material known to those having ordinary skill in the art.

In an exemplary embodiment, fabric outer covering 127 covers ribs 122, 124 and valley 125 as well as planar substrate 110. Additionally, as shown in FIGS. 1, 2, and 4, fabric outer covering 127 can be sewn along a boundary 125a that separates first convex rib 122 from valley 125 and along a boundary 125b that separates the second convex rib 124 from valley 125. Boundaries 125a, 1205b extend generally parallel to longitudinal centerline 126. Sewn boundaries 125a, 125b prevent the granular material 128 inside ribs 122, 124 from migrating into valley 125, thereby maintaining the general shape of each rib 122, 124.

Valley 125 is sized to allow a patient's spine to be accommodated between first and second convex rib 122, 124. In use, mobilization device 100 is typically placed against the patient's back in the location of a particular targeted vertebra that the caregiver desires to mobilize, as shown FIG. 5, with the patient's vertebrae extending between ribs 122, 124 and in valley 125. While the goal may be to mobilize a single vertebra, up to five or six adjacent vertebrae may be mobilized at one time. Also, while FIG. 5 shows a patient lying with his hands behind his head, alternatively, the patient may also lie with his arms crossed in front of his chest.

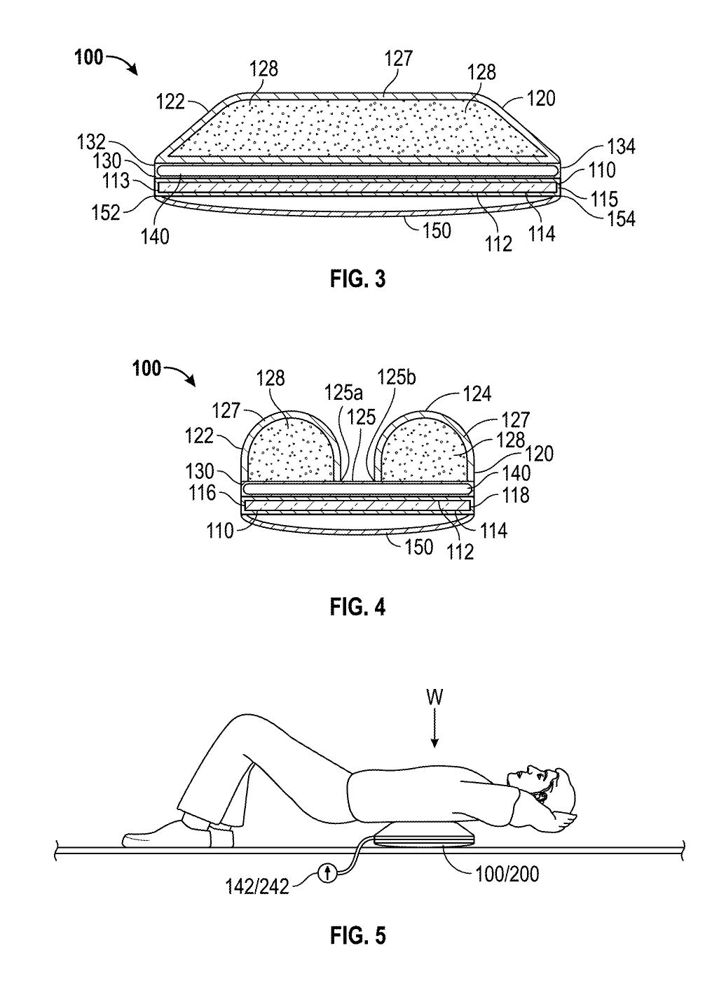

A cavity 130 extends above first face 114 between first face 114 and mobilization portion 120. Cavity 130 is sized to receive a bladder 140 therein. Cavity 130 is defined by an opening formed by fabric outer covering 127 between first face 114 and mobilization portion 120. In an exemplary embodiment, shown in FIG. 3, cavity 130 has a first open end 132 and the second open end 134, distal from first open end 132. In an alternative embodiment, not shown, cavity 130 has a first open end 132 and a closed end 136, distal from first open end 132.

Bladder 140 can be removably inserted into cavity 130. Bladder 140 is in fluid communication with a pressure measurement device 142, such as, for example, an analog or a digital pressure gauge. In an exemplary embodiment, the combination of bladder 140 and pressure measurement device 142 can be a commercially available sphygmomanometer, also known as a blood pressure cuff. A sphygmomanometer can be used because it is a device that is commonly found a caregiver's office and can be readily applied to mobilization device 100. The pressure gauge on the sphygmomanometer typically provides a reading in millimeters of mercury and can be easily read by the caregiver during spinal mobilization. The sphygmomanometer can be inserted into cavity to perform the spinal mobilization and then removed afterward and used to record blood pressures.

Alternatively, bladder 140 can simply be an inflatable device with a pump (not shown) to pump a fluid, such as air, into bladder 140 in order to pressurize bladder 140. Still alternatively, bladder 140 can simply be a pressurized bladder without a pump. Pressurize bladder 140 provides a value on pressure measurement device 142 so that the caregiver can record a baseline value of pressure against mobilization device 100 prior to mobilizing the patient's spine.

Optionally, mobilization device 100 can include an elastic strap 150 that extends across bottom face 114 of planar substrate 110. As shown in FIG. 3, elastic strap 150 includes a first end 152 that is fixedly connected to fabric outer covering 127 proximate to a first lateral side 113 of planar substrate 110 and a second end 154 that is fixedly connected to fabric outer covering 127 proximate to a second lateral side 115 of the substrate 110. The use of strap 150 protects the caregiver's fingers, hand, and wrist joint by keeping them in a more neutral position when treating the patient versus a traditional joint mobilization procedure with a patient in the supine position. Alternatively, if the caregiver and patient are of different genders, use of strap 150 may be omitted if one party is uncomfortable with the caregiver's hand under the patient. This allows the caregiver to cross his/her arms prior to leaning onto the patient's chest in order to provide an interface between the patient and the caregiver.

Additionally, in an exemplary embodiment, a minimum length (longitudinal dimension) of mobilization device 100 is determined so that the caregiver can fit four fingers of one hand between elastic strap 150 and planar substrate 110 in order to hold mobilization device 100 while the caregiver administers treatment to the patient, if the caregivers so desires.

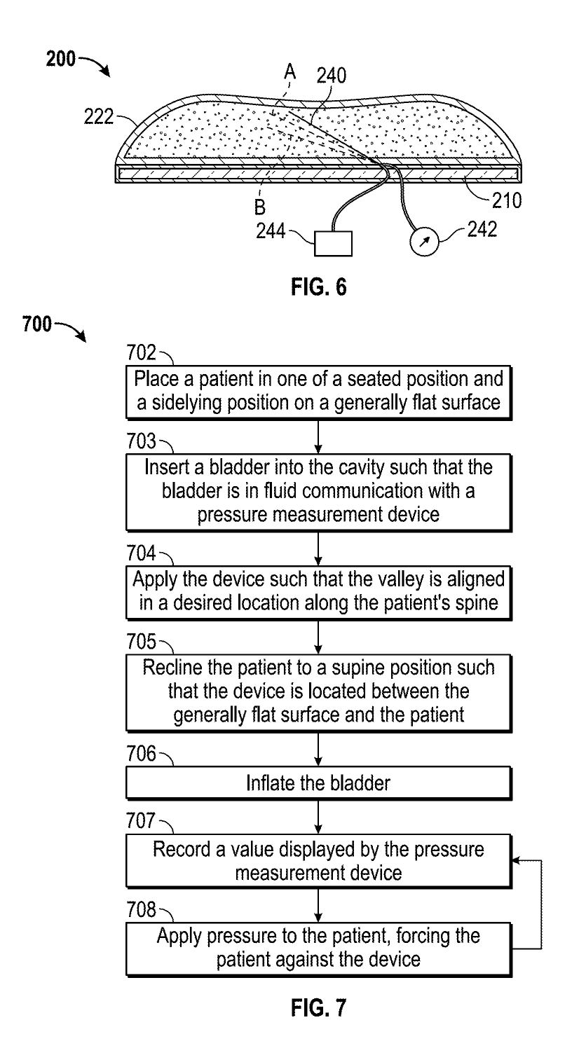

In an alternative embodiment of a mobilization device 200, shown in FIG. 6, instead of bladder 140, a deflection measurement device, such as, for example, a strain gauge 240, can be used to measure the deflection of immobilization device 200 when a caregiver applies pressure to a patient against mobilization device 200.

In an exemplary embodiment, strain gauge 240 can be obliquely attached to a planar substrate 210, with strain gauge 240 extending into at least one rib 222 in a mobilization portion 220. Strain gauge 240 is electrically connected to a display device 242, such as a digital readout, as well as to a power supply 244. While power supply 244 is shown in FIG. 6 as being outside of mobilization device 200, those skilled in the art will recognize the power supply 244 can be integrated inside mobilization device 200.

When a patient lies on mobilization device 200, as shown in FIG. 5, strain gauge 240 is deflected, as shown by dashed line “A”, changing the electrical resistance of strain gauge 240, which generates a first value on display device 242. When the caregiver applies pressure to the patient, strain gauge 240 undergoes an additional deflection, as shown by dashed line “B”, which generates a second value on display device 242. The difference between the first value and the second value correlates to the amount of pressure applied by the caregiver to the patient. The caregiver can note this difference and, in subsequent treatments, knows a target second value by adding the amount of the difference to the first value when the patient lies on mobilization device 200.

Referring to flowchart 700 in FIG. 7, to use mobilization device 100 or 200 to mobilize the patient's spine, in step 702, the patient is first placed in one of a seated position and a sidelying position on a generally flat surface. Referring to mobilization device 100, if bladder 140 is a sphygmomanometer, in step 703, bladder 140 is inserted into cavity 130 in mobilization device 100.

In step 704, the caregiver applies mobilization device 100 such that valley 125 is aligned in a desired location along the patient's spine. The caregiver can insert his/her fingers between elastic strap 150 and planar substrate 110 in order to apply mobilization device 100 at the desired location. Next, in step 705, the caregiver reclines the patient to a supine position such that mobilization device 100 is located between the generally flat surface and the patient, as shown in FIG. 5. As discussed above, the caregiver can hold mobilization device 100 with one hand between elastic strap 150 and planar substrate 110 or, alternatively, the caregiver can remove his/her hand from mobilization device 100.

If bladder 140 is a sphygmomanometer or some other type of inflatable bladder, in step 706, bladder 140 is inflated to a desired value and, in step 707, the value (first value) of pressure measurement device 142 is noted/recorded by the caregiver. If bladder 140 is a pressurized bladder without a pump, the value of pressure measurement device 140 is simply noted/recorded by the caregiver.

Next in step 708, the caregiver applies pressure “W” to the patient, as shown in FIG. 5, forcing the patient against mobilization device 100 until the caregiver determines that the patient's spine has been properly mobilized. At this point, the caregiver notes/records the value (second value) of pressure measurement device 142. The difference between the first value and the second value is an approximate value of the amount of pressure applied by the caregiver to mobilize the patient's spine.

In a subsequent treatment of the patient, the caregiver can note the first value of pressure measurement device 142 prior to applying pressure to the patient and determine, based on the previously noted/recorded difference between the first value and the second value, approximately how much pressure should be required to be applied to the patient in order to mobilize the patient. In addition, if the caregiver notes that a significantly different amount of pressure is required to mobilize the patient's spine in a subsequent treatment as compared to a previous treatment, the caregiver can investigate potential reasons why the different amount of pressure is required. For example, the patient may have gained/lost weight between treatments; the patient's physical condition may have deteriorated; or other reasons may be involved. Additionally, the record of pressure values required to mobilize a spine may be used as part of research to better help target a population that would benefit from such mobilization or to determine a safe amount of pressure to apply for patients who do not normally receive mobilization treatments.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A device for mobilizing a joint comprising:
a flat, planar substrate having:
a first face and an opposing second face; and
a first longitudinal side extending between the first face and the second face; and
a second longitudinal side extending between the first face and the second face, distal from the first longitudinal side;
a mobilization portion extending above the first face and comprising;
a first convex rib extending parallel to the first longitudinal side;
a second convex rib extending along the second longitudinal side and parallel to the first convex rib; and
a valley extending between the first convex rib and the second convex rib along a longitudinal centerline of the flat, planar substrate; and
a cavity extending above the first face, the cavity being defined by an opening between the first face and the mobilization portion and sized to receive a bladder.

2. The device according to claim 1, further comprising a bladder inserted into the cavity, the bladder being in fluid communication with a pressure measurement device.

3. The device according to claim 2 wherein the bladder and the pressure measurement device together comprise a sphygmomanometer.

4. The device according to claim 1, further comprising a rectangular strap extending along the second face, the strap being coupled to the device along a first side and an opposing second side.

5. The device according to claim 1, wherein the first convex rib comprises a flexible material.

6. The device according to claim 5, wherein a granular material is disposed between the flexible material and the first face.

7. The device according to claim 5, wherein the flexible material comprises rubber.

8. A method for mobilizing a spine comprising the steps of:
a. placing a patient in one of a seated position and a sidelying position on a generally flat surface;
b. applying the device according to claim 1 such that the valley is aligned in a desired location along the patient's spine;
c. reclining the patient to a supine position such that the device is located between the generally flat surface and the patient; and
d. applying pressure to the patient, forcing the patient against the device.

9. The method according to claim 8, further comprising the step of inserting a bladder into the cavity prior to performing step c, wherein the bladder is in fluid communication with a pressure measurement device.

10. The method according to claim 9, further comprising, before step d, the step of inflating the bladder.

11. The method according to claim 9, further comprising, after step d, the step of:
e. recording a value displayed by the pressure measurement device.

12. The method according to claim 11, comprising the steps of repeating steps d and e.

13. A device for mobilizing a joint comprising:
a flat, planar substrate having:
a first face and an opposing second face; and
a first longitudinal side extending between the first face and the second face; and
a second longitudinal side extending between the first face and the second face, distal from the first longitudinal side; and
a mobilization portion extending above the first face and comprising;
a first convex rib extending parallel to the first longitudinal side;
a second convex rib extending along the second longitudinal side and parallel to the first convex rib;
a valley extending between the first convex rib and the second convex rib along a longitudinal centerline of the flat, planar substrate; and
a deflection measurement device configured to measure deflection of the mobilization portion toward the planar surface.

14. The device according to claim 13, wherein the deflection measurement device is located inside the mobilization portion.

15. The device according to claim 13, wherein the deflection measurement device comprises a strain gauge.

16. The device according to claim 15, wherein the strain gauge is electrically coupled to a display device.

17. The device according to claim 13, wherein the first convex rib comprises a flexible material.

18. The device according to claim 17, wherein the flexible material comprises a granular material is disposed between the flexible material and the first face.

19. The device according to claim 17, wherein the flexible material comprises rubber.
